(19) United States
(12) Patent Application Publication
(10) Pub. No.: US 2022/0211429 A1
(43) Pub. Date: Jul. 7, 2022

(54) Electrode Assemblies for Measuring Impedance

(71) Applicant: MAGNETO THROMBECTOMY SOLUTIONS LTD., Or Yehuda (IL)

(72) Inventors: Yuval Taff, Tel Aviv (IL); Danil Yarovinski, Bat-Yam (IL); Gal Stern, Tel Aviv (IL)

(21) Appl. No.: 17/700,575

(22) Filed: Mar. 22, 2022

Related U.S. Application Data
(63) Continuation-in-part of application No. 15/859,776, filed on Jan. 2, 2018, now Pat. No. 11,311,332.
(60) Provisional application No. 62/442,470, filed on Jan. 5, 2017.
(60) Provisional application No. 62/519,185, filed on Jun. 14, 2017.

(51) Int. Cl. A61B 18/14

(52) U.S. Cl. CPC A61B 18/1492; A61B 2018/00083

(57) ABSTRACT

An electrode assembly includes an electrically-conductive wire configured for insertion into a blood vessel of a subject, an electrode surrounding the wire, and a discontinuous electrically-insulating cover disposed between the wire and the electrode such that the wire lies radially opposite the electrode at a break in the discontinuous electrically-insulating cover. Other embodiments are also described.

### CROSS-REFERENCE TO RELATED APPLICATIONS

The present application is a continuation-in-part of U.S. patent application Ser. No. 15/859,776, published as US Patent Application Publication 2018/0116717, filed Jan. 2, 2018, which claims the benefit of (i) U.S. Provisional Application 62/442,470, entitled “Thrombectomy device,” filed Jan. 5, 2017, and (ii) US Provisional Application 62/519,185, entitled “Electric thrombectomy device,” filed Jun. 14, 2017. The respective disclosures of the aforementioned applications are incorporated herein by reference.

### FIELD OF THE INVENTION

The present invention relates to the field of medical devices, particularly devices for locating and treating thrombi in blood vessels.

### BACKGROUND

An occlusion of a blood vessel may include a thrombus, an arteriosclerotic lesion, or any other combination of blood, fat, cholesterol, plaque, and/or foreign materials originating from outside the body.

US Patent Application Publication 2004/0073243 describes devices and methods for removing an obstruction from a blood vessel. The devices are deployed in a collapsed condition and are then expanded within the body. The devices are then manipulated to engage and remove the obstruction.

U.S. Pat. No. 6,855,143 describes electrosurgical apparatus and methods for maintaining patency in body passages subject to occlusion by invasive tissue growth. The apparatus includes an electrode support disposed at a shaft distal end having at least one active electrode arranged thereon, and at least one return electrode proximal to the at least one active electrode. In one embodiment, a plurality of active electrodes each comprising a curved wire loop portion are sealed within a distal portion of the electrode support.

U.S. Pat. No. 8,197,478 describes an apparatus and method for electrically induced thrombosis. The surgical device includes a first electrode and a second electrode. The first electrode is for placement adjacent to, near, or within a treatment site of a patient. The second electrode can be movable with respect to the first electrode. When the electrodes are charged by an electricity source, negatively charged blood components are attracted to the positively charged electrode while being repelled from the negatively charged electrode. Due to the electric potential between the adjacent electrodes, thrombosis is induced. The negatively charged blood and components form a thrombus or a clot adjacent to the positively charged electrode. The surgical device can be used to induce the otherwise natural process of thrombosis. When the surgical device is used in a treatment site such as a puncture or incision, the thrombosis can seal the opening created by the treatment site.

U.S. Pat. No. 6,554,827 describes an RF ablation system including a catheter and an RF electrode that delivers RF electrical energy to the site of an occlusion. The system includes a mechanism for minimizing the likelihood that RF electrical energy will be applied directly to the vessel wall. In one embodiment of the invention, the catheter includes a number of tissue expanding jaws to engage a vessel wall and push the vessel wall away from the electrode to shield vessel walls from the electrode. In yet another embodiment of the invention, the electrode has a spiraled distal end with a radius that is larger than the radius of the catheter, such that the electrode engages the vessel wall and pushes the wall away from a conducting portion of the electrode. The portion of the electrode that engages the vessel wall is preferably coated with an insulating material to prevent delivery of RF electrical energy directly to the vessel wall.

U.S. Pat. No. 7,993,334 describes tissue ablation probes, systems, and methods for treating tissue (e.g., a tumor). The probe comprises an inner probe shaft, and an outer probe shaft disposed around the inner probe shaft. The outer probe shaft has a distal portion fixedly mounted to the inner probe shaft, and a proximal portion rotatably mounted to the inner probe shaft. The probe further comprises a coiled ablation electrode disposed between the proximal portion and the distal portion. The electrode is configured for unwinding when the proximal portion rotates about the inner probe shaft in one direction, thereby placing the electrode in a radially expanded geometry, and configured for winding when the proximal portion rotates about the inner probe shaft in another opposite direction, thereby placing the electrode in a radially collapsed geometry.

U.S. Pat. No. 8,496,653 describes a catheter and catheter system that can use energy tailored for remodeling and/or removal of target material along a body lumen, often of a thrombus from a blood vessel of a patient. An elongate flexible catheter body with a radially expandable structure may have a plurality of electrodes or other energy delivery surfaces. The electrode structures may be radially inwardly oriented and/or supported in cantilever to facilitate advancing the electrodes.

Co-assigned U.S. Pat. No. 10,028,782 to Orion, whose disclosure is incorporated herein by reference, describes a flexible catheter device capable of being introduced into body passages and withdrawing fluids therefrom or introducing fluids thereinto, and which includes electrodes configured to apply electrical signals in the body passage for carrying out a thrombectomy, wherein one of said electrodes is designed to contact the thrombus material and remove it or dissolve it, and wherein the electrical voltage signals include a unipolar pulsatile voltage signal.

Co-assigned US Patent Application Publication 2019/0262069 to Taff et al., whose disclosure is incorporated herein by reference, describes an apparatus for removal of a thrombus from a body of a subject including an electrically-insulating tube, which includes a distal end having a circumferential wall that is shaped to define one or more perforations and is configured for insertion into the body of the subject. The apparatus further includes an outer electrode disposed over the distal end of the electrically-insulating tube and configured to lie at least partly within the thrombus while the electrically-insulating tube is inside the body of the subject, and an inner electrode configured to lie, within the tube, opposite the perforations, while the outer electrode lies at least partly within the thrombus. The outer electrode is configured to attract the thrombus while the outer electrode lies at least partly within the thrombus and the inner electrode lies opposite the perforations, when a positive voltage is applied between the outer electrode and the inner electrode such that electric current flows through the perforations.

Co-assigned US Patent Application Publication 2022/0071697 to Taff et al., whose disclosure is incorporated herein by reference, describes an apparatus for treating a blockage in a body of a subject. The apparatus includes a tube configured for insertion into the body and shaped to define a first lumen, and a second lumen having a distal opening. The apparatus further includes a pair of electrodes configured to apply an electric current to the blockage upon application of a voltage between the electrodes, the pair including an outer electrode wrapped around the tube and an inner electrode configured to pass through the first lumen.

### SUMMARY OF THE INVENTION

There is provided, in accordance with some embodiments of the present invention, an electrode assembly, including an electrically-conductive wire configured for insertion into a blood vessel of a subject, an electrode surrounding the wire, and a discontinuous electrically-insulating cover disposed between the wire and the electrode such that the wire lies radially opposite the electrode at a break in the discontinuous electrically-insulating cover.

In some embodiments, the electrode is shaped to define a helix.

In some embodiments, the electrode includes a mesh.

In some embodiments, the electrode is coupled to the discontinuous electrically-insulating cover.

In some embodiments, a length of the break is less than 5 mm.

In some embodiments, the electrode assembly further includes a reinforcing tube disposed around the discontinuous electrically-insulating cover.

In some embodiments, the reinforcing tube is proximal to the break.

In some embodiments, the reinforcing tube is distal to the break.

In some embodiments, the electrode assembly further includes another electrode coupled to the reinforcing tube.

In some embodiments, the break includes a perforation in the discontinuous electrically-insulating cover.

In some embodiments, a surface area of the perforation is less than 0.8 mm².

There is further provided, in accordance with some embodiments of the present invention, an apparatus including the electrode assembly and a treatment device. The treatment device includes a treatment element configured to treat an occlusion in the blood vessel, and an electrically-insulating tube configured to facilitate delivery of the treatment element to the occlusion by advancing over the electrode assembly.

In some embodiments,

the occlusion includes a thrombus,

a distal portion of the electrically-insulating tube is shaped to define one or more apertures, and

the treatment element includes a treatment electrode surrounding the distal portion of the electrically-insulating tube and configured to attract the thrombus when a signal is applied between the treatment electrode and any electrically-conductive element disposed within the electrically-insulating tube, by virtue of an electrical current flowing through the apertures.

In some embodiments,

the electrically-insulating tube is shaped to define a first lumen and a second lumen,

the treatment element further includes a return electrode,

the signal is applied between the treatment electrode and the return electrode while the return electrode is disposed within the first lumen, and

the electrically-insulating tube is configured to advance over the electrode assembly while the electrode assembly is disposed within the second lumen.

In some embodiments, the electrically-insulating tube is shaped to define a lateral window opening into the second lumen and disposed between a proximal end of the treatment electrode and a distal end of the treatment electrode.

There is further provided, in accordance with some embodiments of the present invention, a method including inserting, into a blood vessel of a subject, an electrode assembly including a wire, an electrode surrounding around the wire, and a discontinuous electrically-insulating cover disposed between the wire and the electrode such that the wire lies radially opposite the electrode at a break in the discontinuous electrically-insulating cover. The method further includes, subsequently to inserting the electrode assembly, moving the electrode assembly through the blood vessel while monitoring a signal between the electrode and the wire, which results from ions flowing between the wire and the electrode via the break. The method further includes treating an occlusion in the blood vessel responsively to the signal.

In some embodiments, treating the occlusion includes:

responsively to the signal, positioning a treatment device, which includes an electrically-insulating tube, by advancing the electrically-insulating tube over the electrode assembly; and

using the treatment device, treating the occlusion.

In some embodiments,

the occlusion includes a thrombus,

the signal is a first signal,

a distal portion of the electrically-insulating tube is shaped to define one or more apertures,

the treatment device further includes a treatment electrode surrounding the distal portion of the electrically-insulating tube such that the apertures are disposed between a proximal end of the treatment electrode and a distal end of the treatment electrode, and

treating the thrombus includes attracting the thrombus to the treatment electrode by applying a second signal between the treatment electrode and any electrically-conductive element disposed within the electrically-insulating tube, by virtue of an electric current flowing through the apertures.

In some embodiments, treating the thrombus further includes:

subsequently to a start of the second signal, withdrawing the electrically-insulating tube and the electrode assembly from the blood vessel while the break in the discontinuous electrically-insulating cover is aligned with at least one of the apertures; and

while withdrawing the electrically-insulating tube and the electrode assembly, monitoring the first signal.

In some embodiments, the electrically-conductive element is selected from the group of elements consisting of: the wire, the electrode, and another electrically-conductive element disposed over the wire.

In some embodiments,

the electrically-insulating tube is shaped to define a first lumen and a second lumen,

the apertures open into the first lumen,

the electrically-conductive element includes a return electrode disposed within the first lumen, and

advancing the electrically-insulating tube over the electrode assembly includes advancing the electrically-insulating tube over the electrode assembly while the electrode assembly is disposed within the second lumen.

In some embodiments, the electrically-insulating tube is shaped to define a lateral window opening into the second lumen and disposed between a proximal end of the treatment electrode and a distal end of the treatment electrode, and treating the thrombus further includes:

subsequently to a start of the second signal, withdrawing the electrically-insulating tube and the electrode assembly from the blood vessel while the break in the discontinuous electrically-insulating cover is aligned with the lateral window; and

while withdrawing the electrically-insulating tube and the electrode assembly, monitoring the first signal.

In some embodiments,

the occlusion includes a thrombus,

the signal is a first signal, and

treating the thrombus includes:
positioning the electrode assembly responsively to the first signal; and
attracting the thrombus to the electrode by applying a second signal between the electrode and the wire.

In some embodiments, the method further includes generating the signal by applying a voltage or current between the electrode and the wire.

In some embodiments,

the electrode assembly further includes a pair of other electrodes, and

the method further includes generating the signal by applying a voltage or current between the pair of other electrodes.

The present invention will be more fully understood from the following detailed description of embodiments thereof, taken together with the drawings, in which:

### BRIEF DESCRIPTION OF THE DRAWINGS

FIG. 1 is a schematic illustration of an apparatus for removal of a thrombus from a body of subject, in accordance with some embodiments of the present invention;

FIGS. 2-5 are schematic illustrations of electrode assemblies, in accordance with some embodiments of the present invention;

FIGS. 6-7 are schematic illustrations of thrombectomy devices guided by an electrode assembly, in accordance with some embodiments of the present invention; and

FIG. 8 is a flow diagram for a method for treating a thrombus, in accordance with some embodiments of the present invention.

### DETAILED DESCRIPTION OF EMBODIMENTS

### Overview

In some cases, it may be difficult to locate an occlusion, such as a thrombus or an arteriosclerotic lesion, within a blood vessel.

To address this challenge, embodiments of the present invention provide various electrode assemblies configured to measure impedance inside the blood vessel. Given that the impedance of the occlusion will generally vary from that of the surrounding tissue, the location of the electrode assembly at which a change in impedance is observed may be assumed to be the location of the occlusion. Moreover, in some cases, the type and/or composition of the occlusion may be inferred from the impedance. For example, a lower impedance may indicate a thrombus composed mainly of plasma, whereas a higher impedance may indicate a thrombus composed mainly of red blood cells. Advantageously, knowing the type and/or composition of the occlusion may facilitate more effective treatment of the occlusion.

Each electrode assembly comprises a wire, an electrode (such as a coil, a mesh, or a tube) surrounding the wire, and an insulating material interposing between the wire and the electrode along most of the length of the wire. To measure the impedance, a voltage or current is applied between the electrode and wire or between a separate pair of electrodes near the electrode and wire, and the resulting current or voltage between the electrode and wire is measured.

In some embodiments, the insulating material is discontinuous, such that the electrode lies radially opposite the wire at at least one gap in the insulating material. The gap facilitates the impedance measurements, in that electric current may flow between the electrode and the wire via the gap.

If the occlusion includes a thrombus, the electrode assembly itself may be used to remove the thrombus. For example, a positive voltage may be applied between the electrode and the wire, such that the negatively-charged thrombus becomes electrostatically attached to the positively-charged electrode. Subsequently, the electrode assembly, together with the thrombus, may be withdrawn.

Alternatively, a separate thrombectomy device may be advanced over the electrode assembly and then used to capture or dissolve the thrombus. The thrombectomy device may comprise a treatment electrode surrounding an insulative tube. A positive voltage may be applied between the treatment electrode and any electrically-conductive element with the tube, such as the electrode or wire belonging to the electrode assembly or a separate return electrode, such that the thrombus becomes electrostatically attached to the treatment electrode.

If the occlusion does not include a thrombus (e.g., if the occlusion includes an arteriosclerotic lesion), any other suitable treatment device, such as a stent-delivery device, may be advanced over the electrode assembly and then used to treat the occlusion.

### Apparatus Description

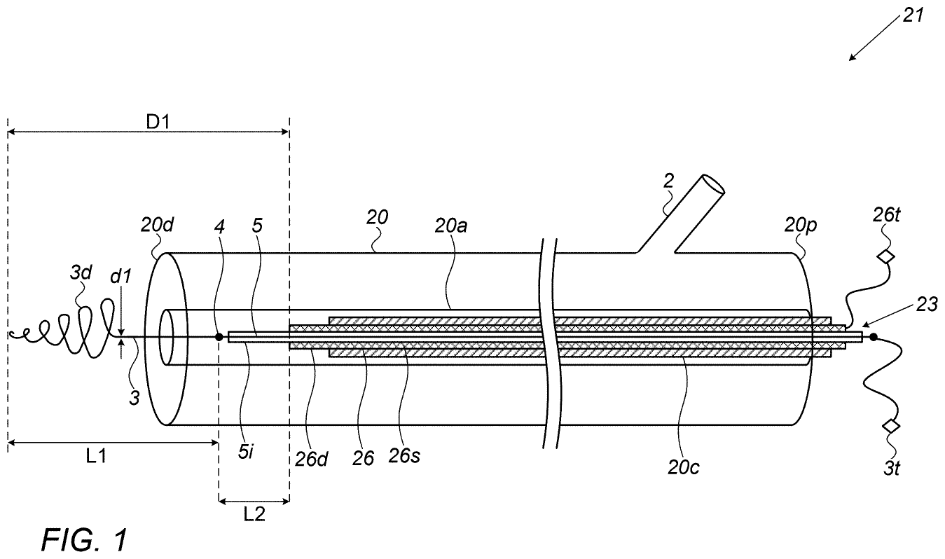

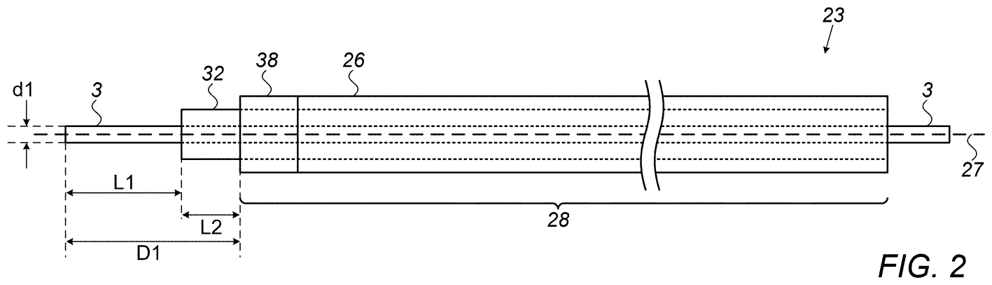

Reference is initially made to FIG. 1, which is a schematic illustration of apparatus 21 for removal of a thrombus from a body of subject, in accordance with some embodiments of the present invention. FIG. 1 generally corresponds to FIG. 2 of US Patent Application Publication 2011/0301594, whose disclosure is incorporated herein by reference.

Apparatus 21 comprises a catheter 20, which has a proximal end 20p and a distal end 20d, and which is shaped to define a lumen 20a. Following the introduction of catheter 20 into the vascular system of the subject, e.g., using standard angiographic catheterization techniques, an electrode assembly 23 is passed through lumen 20a, and is subsequently used to remove a thrombus from the vascular system, as described in detail hereinbelow. In some embodiments, catheter 20 further comprises a proximal, lateral port 2 for withdrawing any debris (e.g., thrombus fragments) generated during the treatment process, using a syringe (not shown) or any other device suitable for this purpose. Alternatively or additionally, a second catheter, passing over catheter 20, may be positioned proximally to the thrombus, and subsequently used to aspirate such debris. Alternatively or additionally, a net disposed near the distal end of electrode assembly 23 may be used to catch and remove such debris.

In the particular embodiment shown in FIG. 1, electrode assembly 23 comprises a pair of coaxial electrodes: an inner electrode 3 and an outer electrode 26. Inner electrode 3 comprises a wire having a diameter dl that may have any suitable value, such as between 0.01 and 4 mm.

Inner electrode 3 comprises a distal end 3d configured to contact the thrombus. In some embodiments, distal end 3d is straight. Alternatively, as shown in FIG. 1, distal end 3d may be curly, or may have any other suitable shape that increases the contact area between the electrode and the thrombus, relative to a straight distal end. For example, the surface of distal end 3d (or of the entire inner electrode) may comprise a plurality of protrusions, or bumps, which increase the surface area available for contact with the thrombus. Alternatively or additionally, distal end 3d (or the entire inner electrode) may be curved, such as to decrease the likelihood that the electrode will damage tissue of the subject.

In some embodiments, inner electrode 3 is connected at its proximal end, at a connection point 4, to another wire 5, which passes through lumen 20a to proximal end 20p of the catheter. In other embodiments, instead of wire 5, inner electrode 3 extends through the lumen of the catheter, to the proximal end of the catheter.

Typically, an electrically-isolating material separates the inner electrode from the outer electrode, such that the inner and outer electrodes are electrically isolated from one another. For example, an electrically-isolating layer 5i may cover wire 5, with outer electrode 26, in turn, covering electrically-isolating layer 5i. For example, as shown in FIG. 1, outer electrode 26 may comprise a multi-stranded wire, comprising a plurality of electrically-conducting strands 26s that are braided over, or wrapped around, electrically-isolating layer 5i. In some embodiments, an electrically-isolating cover 20c covers most of the outer electrode, such that only a distal portion 26d of the outer electrode remains exposed. In some embodiments, distal portion 26d is between 7 and 25 mm long, e.g., around 15 mm long.

Typically, the inner electrode—or the exposed portion of the inner electrode, which is the portion of the inner electrode not covered by electrically-isolating layer 5i—has a length L1 that is between 0.1 and 150 mm (e.g., between 5 and 50 mm, such as between 5 and 25 mm). Alternatively, length L1 may have any other suitable value. The distal end of the inner electrode is typically blunt, to help prevent any damage to the lumen through which the inner electrode is passed.

Wire 5 terminates, at its proximal end, at a first terminal 3t. Similarly, the outer electrode terminates, at its proximal end, at a second terminal 26t. Upon the inner electrode contacting the thrombus, a positive voltage is applied between the inner electrode and the outer electrode, via first terminal 3t and second terminal 26t. The positive voltage facilitates a removal of the thrombus, by causing the negatively-charged thrombus to become attached to the inner electrode. Subsequently to the attachment of the thrombus to the inner electrode, apparatus 21 is withdrawn from the blood vessel.

In general, the voltage signal applied to the terminals may have any suitable form, such as any of the forms described in US Patent Application Publication 2011/0301594, whose disclosure is incorporated herein by reference. For example, the voltage signal may be a periodic signal that includes a sequence of pulses, each of these pulses, for example, being shaped as the positive half-wave of a sinusoidal signal, or having a trapezoidal shape. Alternatively, the voltage signal may be a direct current (DC) voltage signal.

Although the amplitude of the voltage may have any suitable value, this amplitude is typically between 1 and 100 V, such as between 1 and 50 V, e.g., between 4 and 40 V. Such an amplitude is large enough to be effective, yet small enough so as to avoid damaging the tissue near the thrombus. For example, as described in US Patent Application Publication 2011/0301594 with reference to FIG. 1D thereof, each trapezoidal pulse of the applied voltage signal may (i) linearly ramp up from ground level (0 V) to an amplitude of around 40 V, over a time period of around 5 milliseconds, (ii) remain constant over a time period of around 5 milliseconds, and then (iii) linearly ramp down to ground level over a time period of around 5 milliseconds. Before the beginning of the subsequent pulse, the voltage may remain at ground level for another time period of around 5 milliseconds.

In general, the applied voltage signal, if pulsatile, may have any suitable frequency, such as between 0.1 Hz to 100 MHz, e.g., around 50 Hz, as in the example immediately above. Typically, the voltage is applied such that a current having an amplitude of between 0.1 and 4 mA (e.g., 1-3 mA) is passed between the inner and outer electrodes.

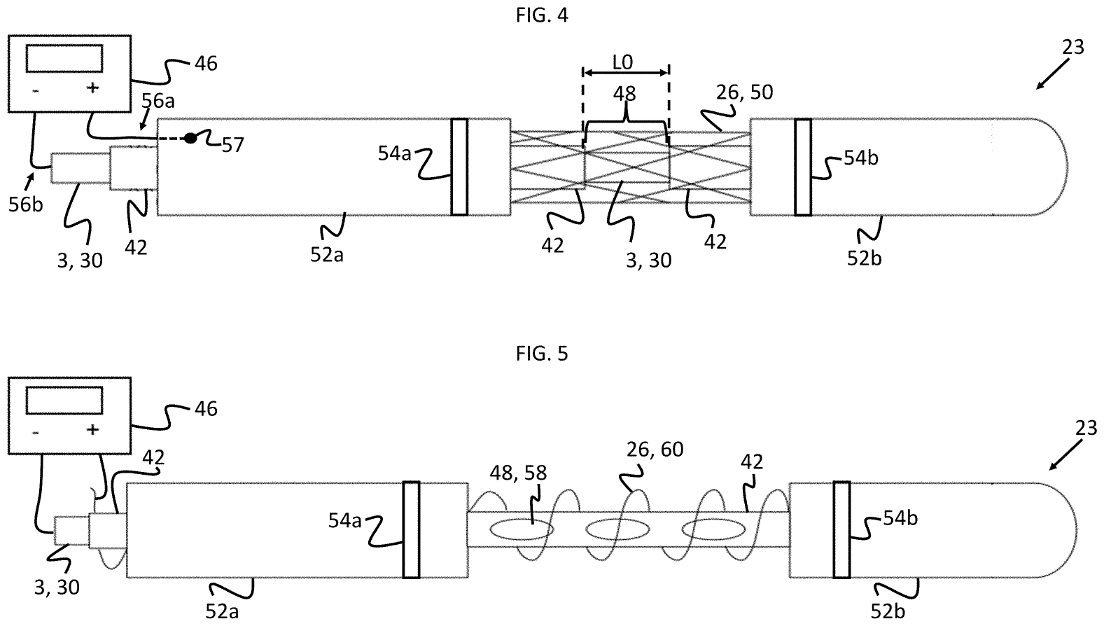

The voltage is applied by a power source 46, shown in FIGS. 4-7. In some embodiments, the power source is current-regulated, e.g., to between 0.1 and 4 mA. In other embodiments, the power source is voltage-regulated, e.g., to between 1 and 50 V. Typically, the voltage is applied for a duration of more than one second, to facilitate attachment of the thrombus to the inner electrode, but less than 10 minutes, to prevent risk to the patient. For example, the duration may be more than 5 seconds but less than 5 minutes, e.g., more than 10 seconds but less than 2 minutes.

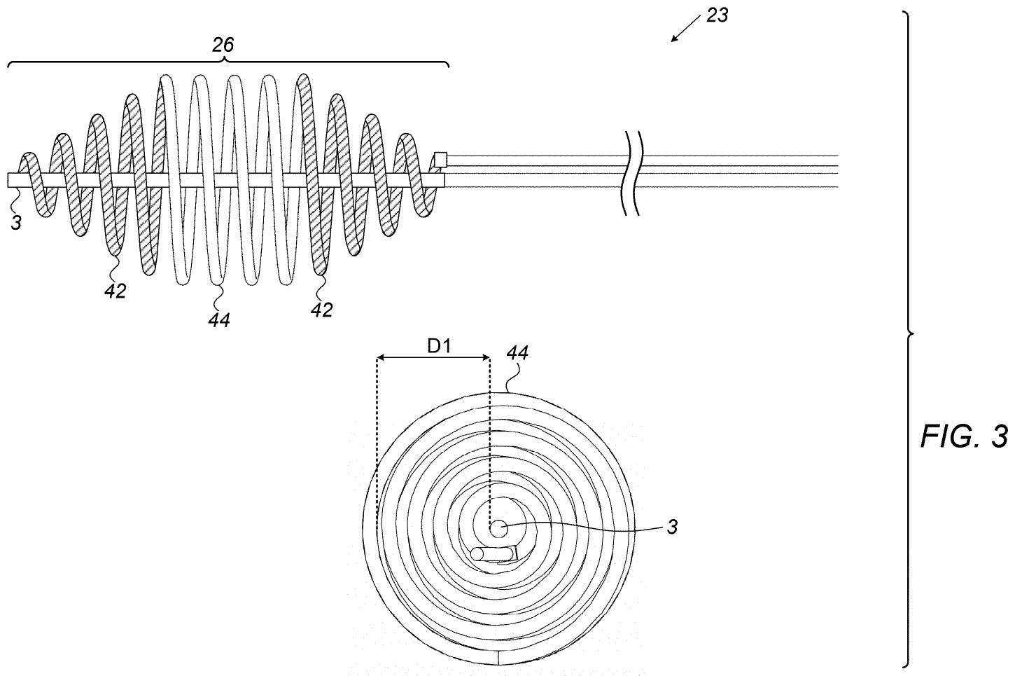

Typically, the voltage is applied while the inner electrode is in contact with the thrombus, and while the outer electrode is inside the body of the subject, e.g., within the catheter lumen, but not in contact with the thrombus. (Notwithstanding the above, it is noted that in some embodiments, e.g., as described below with reference to FIG. 3, both of the electrodes may contact the thrombus.) For example, prior to applying the voltage, the electrode assembly may be advanced such that the inner electrode pierces the thrombus (i.e., passes through the thrombus in contact therewith). Alternatively, catheter 20, with the two electrodes appropriately positioned within the catheter lumen, may be advanced through the thrombus and then withdrawn from over the inner electrode, such that the inner electrode is positioned within the thrombus.

In some cases, it may be advantageous for the position of the catheter to remain as distal as possible during the application of the voltage, to facilitate the collection of any bubbles or debris generated during the procedure. Hence, the outer electrode, and even the inner electrode and the thrombus with which it is in contact, may be partly or fully contained within the catheter lumen while the voltage is applied. For example, following, or together with, the advancement of the electrode assembly as described in the paragraph above, the catheter may also be advanced, such that the outer electrode and/or the inner electrode are contained with the catheter lumen during the subsequent application of the voltage.

Typically, while the voltage is applied, the respective distal tips of the electrodes are spaced apart from each other by a distance D1 of between 1 and 100 mm, such as between 2 and 30 mm. Such a distance facilitates suitable electrical conductivity between the electrodes via the blood at the treatment site, while maintaining the outer electrode at a sufficient distance from the thrombus such as to prevent contact of the outer electrode with the thrombus. Alternatively, distance D1 may be less than 1 mm (in which case the outer electrode may contact the thrombus), or more than 100 mm.

In some embodiments, the separation distance L2 between the inner electrode and the outer electrode (i.e., the distance between the proximal tip of the inner electrode and the distal tip of the outer electrode) is relatively small, so as to reduce the amount of electric current that passes through the tissue surrounding the blood vessel in which the thrombus is located. For example, assuming the total diameter of (i) the blood vessel, and (ii) the tissue surrounding the blood vessel, is D2, such that the total transverse cross-sectional area A2 of the blood vessel and the surrounding tissue is π*(D2/2)², L2 may satisfy the relation L2*(1 mm)<<A2, where “<<” implies “at least one order of magnitude smaller than.” (The above assumes L2 is given in mm, and A2 in mm².) In some embodiments, L2 is even smaller, in that L2 satisfies the relation L2*(1 mm)<<A1, where A1 is the transverse cross-sectional area of the blood vessel.

In general, the ease of manufacture increases with L2. Hence, for ease of manufacture, embodiments of the present invention typically set L2 in accordance with the blood-vessel dimensions, rather than always making L2 as small as possible. In other words, for a relatively large blood vessel, since it may not be necessary to have such a small separation between the electrodes, a larger separation distance may be used, relative to a smaller blood vessel. Some embodiments of the present invention define a range of suitable separations for each particular application, where the upper limit of the range is one order of magnitude less than A1/(1 mm), and the lower limit of the range is two orders of magnitude smaller than A1/(1 mm).

For example, in neurovascular applications, a relatively large vessel may be around 6 mm in diameter, such that the vessel has a cross-sectional area of around 30 mm². Hence, for such applications, distance L2 may be between 0.3 mm and 3 mm. Smaller vessels, such as in the more distal segments of the middle cerebral artery (MCA) in the brain, have a cross-sectional area of around 7 mm². Hence, for such applications, L2 may be between 0.07 mm and 0.7 mm. For the treatment of other conditions, such as deep vain thrombosis, pulmonary embolisms, or coronary artery occlusions, L2 may likewise be set in accordance with the blood-vessel diameter (or cross-sectional area), as described above.

Typically, the electrodes are made of different respective conductive metals, with the inner electrode typically having a higher electronegativity than the outer electrode. For example, the inner electrode may be made of gold or platinum, while the outer electrode may be made of titanium or stainless steel.

(It is noted that, in the context of the present description and claims, an electrode may be considered to be “made of” a particular material, even if it is only coated by this material. For example, an electrode made of titanium may comprise any suitable material that is coated by a layer of titanium.)

In some embodiments, apparatus 21 comprises radiopaque markers, which facilitate visualization of the apparatus using x-ray imaging. For example, one or more radiopaque gold rings or coatings may cover a portion of the outer electrode, if the outer electrode is made of titanium or any other material that is generally not radiopaque.

In some embodiments, apparatus 21 comprises a balloon, disposed proximally to the inner electrode. Prior to the inner electrode contacting the thrombus, the balloon is inflated so as to center the inner electrode relative to the thrombus. The inner electrode may then pass through the center of the thrombus, thus increasing the effectiveness of the subsequently applied voltage.

In some embodiments, the impedance between the inner electrode and outer electrode is measured as the inner electrode is advanced through the blood vessel. This measured impedance indicates the extent to which the inner electrode is near (or in contact with) the thrombus.

In some embodiments, to measure the impedance, a voltage, which is lower than the voltage applied for treatment, is applied between the electrodes, and the resulting current is then measured. The impedance is then the applied voltage divided by the measured current. In other embodiments, the impedance is measured by passing a low current between the electrodes, and then measuring the resulting voltage. The impedance is then the applied current divided by the measured voltage. (Since, however, the actual value of the impedance is not necessarily of interest, the impedance may be “measured” by measuring the current or voltage, even without computing the actual impedance value. For example, once the measured current or voltage reaches a predefined amplitude, it may be ascertained that the inner electrode is in maximum contact with the thrombus.)

Notwithstanding FIG. 1, it is noted that any embodiment of electrode assembly 23 described herein may be passed directly through the blood vessel, without being passed through catheter 20.

Reference is now made to FIG. 2, which is a schematic illustration of electrode assembly 23 in accordance with other embodiments of the present invention.

In FIG. 2, outer electrode 26 is tubular, in that electrode assembly 23 comprises a tube 28 comprising the outer electrode. The outer electrode is coaxial with inner electrode 3 in that the two electrodes share a common longitudinal axis 27. In particular, inner electrode 3 passes through the lumen of a tubular insulator 32, which in turn passes through the lumen of outer electrode 26. Insulator 32 is thus disposed proximally to the exposed portion of the inner electrode, and the outer electrode, in turn, is disposed proximally to the exposed portion of the insulator.

In some embodiments, inner electrode 3, tubular insulator 32, and outer electrode 26 are fixed in place relative to each other. In other embodiments, at least one of these elements is slidable with respect to the others. For example, the inner electrode may be slidable within the tubular insulator, and/or the outer electrode may be slidable over the tubular insulator. Thus, for example, prior to applying the treatment voltage or measuring the impedance within the blood vessel, the outer electrode may be advanced over the insulator until the distance D1 between the respective distal tips of the electrodes is less than a predefined target (such as 100 mm or 30 mm, as described above with reference to FIG. 1), and/or until the distance between the two electrodes (i.e., the exposed length of insulator 32) is less than a predefined target separation distance L2 (such as 3 mm or 0.7 mm). Alternatively or additionally, prior to applying the voltage, the inner electrode may be advanced through the lumen of the insulator until the distance between the respective distal tips of the electrodes reaches a predefined target, and/or until the distance between the two electrodes reaches a predefined target.

In some embodiments, outer electrode 26 is shaped to define only the distal portion of the wall of tube 28 (i.e., the outer electrode does not extend to the proximal end of tube 28), and is therefore connected to the proximal end of electrode assembly 23 via a wire. Alternatively or additionally, inner electrode 3 may not extend to the proximal end of the electrode assembly; rather, a wire, passing through the lumen of insulator 32, may connect inner electrode 3 to the proximal end of the electrode assembly.

In some embodiments, the exposed portion of the inner electrode is straight, as shown in FIG. 2. In other embodiments, the exposed portion of the inner electrode is curved so as to decrease the likelihood that the electrode will damage tissue of the subject.

As described above with reference to FIG. 1, radiopaque markers may be disposed at any suitable location on electrode assembly 23. For example, FIG. 2 shows an embodiment in which the distal portion of the outer electrode comprises a radiopaque marker 38 comprising a ring of radiopaque material.

In some embodiments, a second tube, concentric with tube 28, is disposed within, or around the outside surface of, tube 28. The second tube may be radiopaque, thus facilitating visibility of the electrode assembly under fluoroscopy, and/or may impart particular mechanical properties (e.g., rigidity) to the electrode assembly.

Reference is now made to FIG. 3, which is a schematic illustration of electrode assembly 23 in accordance with other embodiments of the present invention.

In some embodiments, outer electrode 26 wraps around inner electrode 3, with a radial gap separating the two electrodes from one another. For example, outer electrode 26 may be shaped to define a helix, and inner electrode 3, which is typically rod-shaped, may pass through the outer electrode along the longitudinal axis of the outer electrode.

In such embodiments, the proximal and distal portions of the outer electrode may be covered by an insulating cover 42, such that only the middle portion 44 of the outer electrode is exposed. Insulating cover 42, which may comprise any suitable biocompatible polymer such as polyether block amide (PEBA), silicone, polyurethane, polyethylene, or polytetrafluoroethene (PTFE), helps prevent unwanted electrical contact between the two electrodes. In some embodiments, to further help prevent contact, middle portion 44 has a greater radius than other portions of the outer electrode.

Typically, the inner electrode passes through the center of the outer electrode, such that the distance D1 between the inner electrode and middle portion 44, which is approximately equal to the radius of middle portion 44, is between 1 and 100 mm, such as between 2 and 30 mm. Alternatively, distance D1 may have any other suitable value.

Typically, the outer electrode is expandable. Prior to applying the treatment voltage, catheter 20 (FIG. 1), which contains both the outer electrode, and the inner electrode in a crimped configuration, is advanced through the thrombus. Subsequently, the catheter is withdrawn from over the two electrodes, such that the outer electrode expands, from the crimped configuration, within the thrombus. Subsequently, a positive voltage is applied between the outer and inner electrodes, causing the thrombus to become attached to the outer electrode. During the application of the voltage, the inner electrode may protrude distally from the outer electrode. Alternatively, the distal end of the inner electrode may remain inside of the outer electrode.

Alternatively or additionally to treating a thrombus, electrode assembly 23 may be used to measure the impedance within the blood vessel, e.g., as described below with reference to FIG. 4.

Reference is now made to FIG. 4, which is a schematic illustration of electrode assembly 23, in accordance with other embodiments of the present invention.

The features of electrode assembly 23 shown in FIG. 4 are similar to those shown in FIG. 3 in several respects.

For example, in both figures, inner electrode 3 comprises an electrically-conductive wire 30 configured for insertion into a blood vessel of a subject, and outer electrode 26 surrounds wire 30. Furthermore, electrically-insulating cover 42, which is discontinuous, is disposed between wire 30 and electrode 26 such that the wire lies radially opposite the outer electrode at a break 48 (also referred to herein as a “gap”) in cover 42. Cover 42 may comprise an insulating tube in which the inner or outer electrode is disposed, or a layer of insulating material that coats the inner or outer electrode. One or both of the electrodes may be coupled to cover 42.

Moreover, in both figures, to facilitate treatment of an occlusion, the impedance between inner electrode 3 and outer electrode 26 may be measured, e.g., as further described below.

The features shown in FIG. 4 also differ from those shown in FIG. 3 in several respects. For example, whereas in FIG. 3 electrode 26 comprises a coil, in FIG. 4 electrode 26 comprises a mesh 50. Also, in FIG. 4, electrode assembly 23 further comprises a proximal reinforcing tube 52a, which is proximal to break 48, and a distal reinforcing tube 52b, which is distal to break 48 and may comprise a closed distal end. Reinforcing tube 52a and reinforcing tube 52b are disposed around cover 42, and provide structural reinforcement (i.e., greater mechanical stability) to the electrode assembly. Optionally, inner electrode 3 and/or outer electrode 26 may be coupled to one or both of the reinforcing tubes.

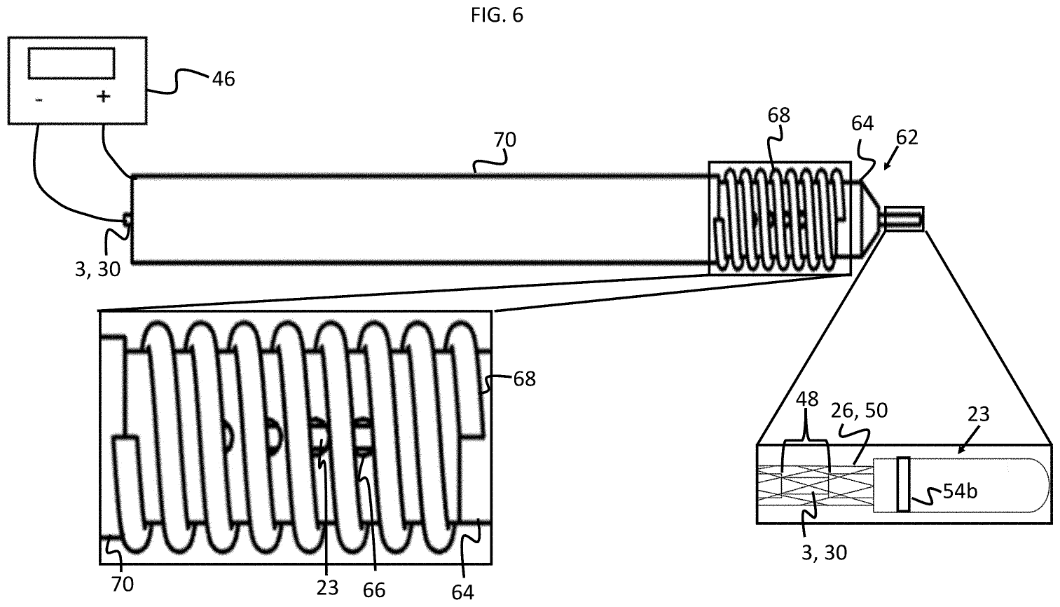

Outer electrode 26 and inner electrode 3 are configured to connect to power source 46 via respective wires 56a and 56b. In some embodiments, as shown in FIG. 4, the connection 57 between wire 56a and outer electrode 26 is inside proximal reinforcing tube 52a. In other embodiments, the outer electrode protrudes from the proximal end of the proximal reinforcing tube, and wire 56a connects to outer electrode 26 proximally to the proximal reinforcing tube, e.g., as shown in FIG. 5 (described below).

Electrode assembly 23 is inserted into the blood vessel of a subject in which an occlusion is located. Subsequently, the electrode assembly is moved through the blood vessel while power source 46 applies a voltage (if the power source is voltage-regulated) or a current (if the power source is current-regulated) between the inner and outer electrodes, thus causing ions (i.e., an electric current) to flow between the inner and outer electrodes via break 48. While the voltage or current is applied, a related signal between the two electrodes, referred to below as an impedance-indicating signal, is measured. In particular, if the power source applies a voltage, the current is measured, whereas if the power source applies a current, the voltage is measured. The impedance-indicating signal may be measured by power source 46 or by any other device and displayed on a computer monitor or any other suitable display, which may be connected wiredly or wirelessly to the power source or other device.

Subsequently, the occlusion is treated responsively to the impedance-indicating signal. In particular, the impedance-indicating signal may indicate the location, type, and/or composition of the occlusion. Responsively to this information, the operating physician may choose a treatment element for treating the occlusion, decide on a treatment technique, and/or position the treatment element.

For example, in response to the impedance-indicating signal indicating the location of a thrombus, the electrode assembly may be positioned such that gap 48 is within the thrombus. Subsequently, a treatment signal, such as the treatment voltage described above with reference to FIG. 1, may be applied, by power source 46, between the inner and outer electrodes, thus causing the outer electrode to attract the thrombus.

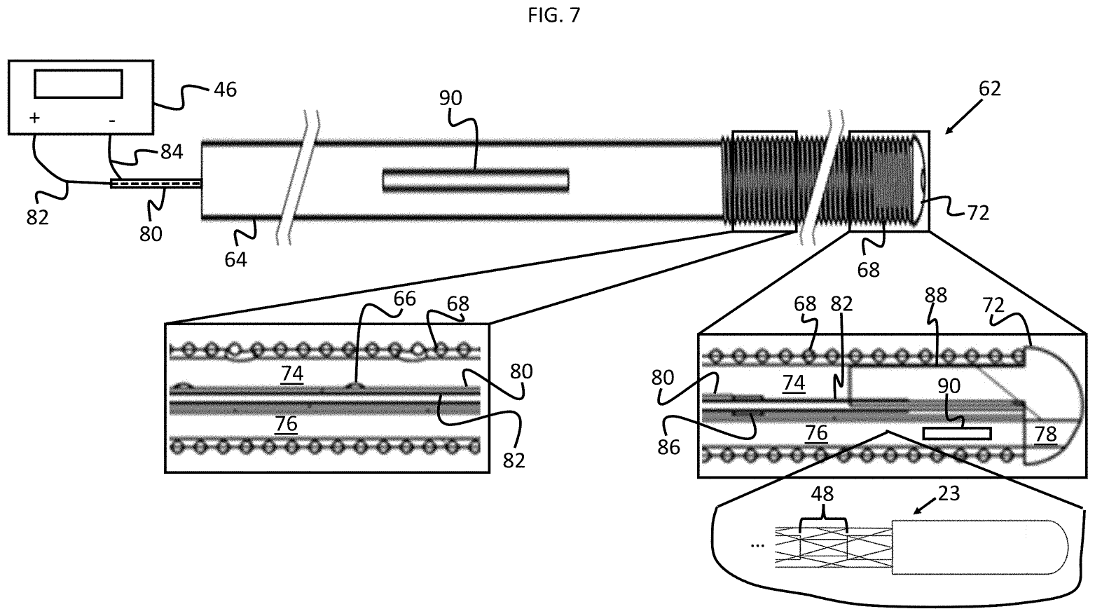

Alternatively, a separate thrombectomy device, which comprises a treatment element and an electrically-insulating tube, may be positioned responsively to the impedance-indicating signal. The treatment element may comprise, for example, a treatment electrode (as described below with reference to FIGS. 6-7), a needle, an ultrasonic transducer, a stent retriever, an aspiration catheter, and/or one or more pincers.

In particular, to facilitate delivery of the treatment element to the thrombus, the electrically-insulating tube may be advanced over the electrode assembly until the distal end of the tube is at or near the thrombus. If the treatment element is not coupled to the tube, the treatment element may be deployed from the tube or passed over the tube. For example, a stent retriever may be deployed from the tube, e.g., such that the proximal end of the retriever is aligned with the proximal end of the thrombus. As another example, an aspiration catheter may be passed over the tube, e.g., until the distal opening of the aspiration catheter is at the proximal end of the thrombus. Subsequently, the treatment element may be used to capture or dissolve the thrombus.

As another example, in response to the impedance-indicating signal indicating the location of an arteriosclerotic lesion, a stent-delivery catheter may be advanced over the electrode assembly, and a stent may then be deployed from the catheter at the location of the lesion.

In some embodiments, electrode assembly 23 further comprises a pair of additional electrodes: an electrode 54a, which is coupled to proximal reinforcing tube 52a or otherwise disposed proximally to gap 48, and another electrode 54b, which is coupled to distal reinforcing tube 52b or otherwise disposed distally to gap 48. For example, electrode 54a may comprise a ring that wraps around proximal reinforcing tube 52a, and electrode 54b may comprise a ring that wraps around distal reinforcing tube 52b. Electrodes 54a and 54b are connected to power source 46 or another power source, e.g., via wires passing through the reinforcing tubes.

In such embodiments, alternatively to generating the impedance-indicating signal by applying a voltage or current between the outer and inner electrodes as described above, the impedance-indicating signal may be generated by applying a voltage or current between the pair of additional electrodes, thus inducing the impedance-indicating signal. Advantageously, this technique for generating the impedance-indicating signal may reduce noise in the impedance-indicating signal by reducing surface effects. Typically, the distance between electrode 54a and break 48, and between electrode 54b and break 48, is less than 50 mm (e.g., less than 10 mm), so as to facilitate using this technique.

In other embodiments, electrode assembly 23 comprises electrode 54a but not electrode 54b, or electrode 54b but not electrode 54a. In such embodiments, electrode 54a or electrode 54b may be used, together with the outer and inner electrodes, to implement any suitable three-electrode impedance-measuring technique known in the art.

Typically, any voltage applied for impedance measurement is between 0.1 mV and 10 V, while any current applied for impedance measurement is between 10 nA and 1 mA. The applied voltage or current may have a frequency between zero and 1 MHz, such as between 1 kHz and 100 kHz.

In other embodiments, the electrode assembly comprises proximal reinforcing tube 52a but not reinforcing tube 52b, distal reinforcing tube 52b but not proximal reinforcing tube 52a, or no reinforcing tube at all. Alternatively, instead of a reinforcing tube, the electrode assembly may comprise a braided shaft, a polymer jacket, a coating, and/or any other reinforcing element. In some embodiments, reinforcing tube 52b (or any alternative distal reinforcing element) is shapeable, or is angled or otherwise pre-shaped to a predefined shape (e.g., a J-shape).

Typically, the length L0 of break 48 is relatively small (e.g., less than 5 mm, such as less than 1 mm), so as to increase the spatial resolution of the impedance measurements.

Typically, to locate the occlusion, a property of the impedance-indicating signal, such as the amplitude of the signal, is compared (automatically or by a user) to a baseline value as the electrode assembly is moved through the blood vessel. (The baseline value may be measured, for example, at a location at which the blood flows unimpeded.) The proximal and distal ends of the occlusion may be assumed to be located where the difference between the value of the property and the value of the baseline crosses a predefined threshold.

Typically, at least one portion of electrode assembly 23 at or near gap 48 is radiopaque, and the electrode assembly is moved through the blood vessel under fluoroscopic imaging. Thus, upon the impedance-indicating signal indicating that gap 48 has reached the occlusion, the occlusion may be assumed to be located at or near the radiopaque portion. For example, outer electrode 26, inner electrode 3, electrode 54a, and/or electrode 54b may be radiopaque. Alternatively or additionally, one or more radiopaque markers may be coupled to any of the aforementioned electrodes and/or reinforcing tubes. Similarly, any separate device used to treat the occlusion typically comprises a radiopaque treatment element (e.g., a radiopaque treatment electrode or stent retriever) and/or one or more suitably-placed radiopaque markers, and the device is deployed under fluoroscopy, such that the treatment element may be positioned properly relative to the occlusion.

Reference is now made to FIG. 5, which is a schematic illustration of electrode assembly 23 in accordance with other embodiments of the present invention.

In some embodiments, break 48 does not extend around the full circumference of the insulating cover, as in FIGS. 3-4. Rather, the break includes a perforation 58 in cover 42. For example, as shown in FIG. 5, cover 42 may be shaped to define multiple perforations 58, which may be arranged in a row (i.e., linearly) as shown in FIG. 5, in a spiral, or in any other suitable pattern. Typically, the surface area of each perforation 58 is relatively small (e.g., less than 0.8 mm², such as less than 0.5 mm²), so as to increase the spatial resolution of the impedance measurements and to inhibit contact of the outer electrode with the inner electrode.

FIG. 5 also differs from FIG. 4 in that, in FIG. 5, outer electrode 26 comprises a helical coil 60. Nevertheless, it is noted that mesh 50 (FIG. 4) may be combined with perforations 58, and coil 60 may be combined with a full-circumference break as shown in FIG. 4.

In some embodiments, for FIGS. 3-5, the electrodes are made of different respective conductive metals, with the outer electrode typically having a higher electronegativity than the inner electrode. For example, the outer electrode may be made of gold or platinum, while the inner electrode may be made of titanium or stainless steel.

Reference is now made to FIG. 6, which is a schematic illustration of a thrombectomy device 62 guided by electrode assembly 23, in accordance with some embodiments of the present invention.

Thrombectomy device 62 comprises an electrically-insulating tube 64, which is typically made of a biocompatible polymer such as polyether block amide (PEBA), silicone, polyurethane, polyethylene, or polytetrafluoroethene (PTFE). Tube 64 is shaped to define a distal opening, which facilitates the advancement of tube 64 over electrode assembly 23 so as to position the thrombectomy device for treatment of the thrombus.

In some embodiments, the distal portion of tube 64 is shaped to define one or more apertures 66, which may be arranged in a row (i.e., linearly) as shown in FIG. 6, in a spiral, or in any other suitable pattern. In addition, the thrombectomy device further comprises a treatment electrode 68 wrapped around the distal portion of tube 64 such that apertures 66 are disposed between the proximal end of the treatment electrode and the distal end of the treatment electrode. Treatment electrode 68 is configured to attract the thrombus, such that the thrombus becomes electrostatically attached to the treatment electrode, when a treatment signal is applied between the treatment electrode and any electrically-conductive element disposed within tube 64, by virtue of an electrical current flowing through apertures 66. The treatment signal may include, for example, the treatment voltage described above with reference to FIG. 1.

For example, as shown in FIG. 6, the treatment signal may be applied between treatment electrode 68 and inner electrode 3. Alternatively, the treatment signal may be applied between the treatment electrode and outer electrode 26 or another electrically-conductive element disposed over the inner electrode, such as electrode 54a (FIGS. 4-5) or electrode 54b.

Following the attachment of the thrombus to the treatment electrode, the thrombectomy device is withdrawn, optionally while the application of the treatment signal continues. Optionally, a suctional force may be applied to the thrombus via apertures 66, thereby facilitating the removal of the thrombus.

In some embodiments, while the thrombectomy device is withdrawn, electrode assembly 23 is also withdrawn, with break 48 being aligned with at least one of apertures 66. During the withdrawal, the power source applies a voltage or current between outer electrode 26 and inner electrode 3, and the resulting impedance-indicating signal is monitored so as to ascertain that no portion of the thrombus disassociated from treatment electrode 68.

In some embodiments, as shown in FIG. 6, power source 46 is connected to the treatment electrode, e.g., via an electrically-conductive tube 70 disposed over tube 64 or via any other suitable electrically-conductive element. In such embodiments, power source 46 applies the treatment signal. For example, after measuring the impedance using the electrode assembly, the positive terminal of the power source may be disconnected from outer electrode 26 and connected, instead, to the treatment electrode. Subsequently, the power source may apply the treatment signal between treatment electrode and inner electrode 3.

Typically, however, the treatment electrode and the portion of electrode assembly 23 that functions as the return electrode are connected to a separate power source. Thus, advantageously, there is no need to modify the wiring during the procedure, and, if required, monitoring of the impedance may continue while the treatment signal is applied.

Reference is now made to FIG. 7, which is another schematic illustration of thrombectomy device 62 guided by electrode assembly 23, in accordance with some embodiments of the present invention.

FIG. 7 differs from FIG. 6 in that, in FIG. 7, tube 64 is shaped to define at least two lumens running alongside one another along the length of the tube: a first lumen 74 and a second lumen 76. Typically, first lumen 74 and second lumen 76 are not in fluid communication with one another within the tube.

Typically, first lumen 74 is closed at its distal end, whereas second lumen 76 is open. For example, device 62 may comprise an end cap 72 that seals the distal end of first lumen 74 without sealing the distal end of second lumen 76. For example, end cap 72 may cap the distal end of tube 64, but may be shaped to define an aperture 78 aligned with second lumen 76.

Device 62 further comprises a return electrode 80, which is configured to pass through first lumen 74. In some embodiments, return electrode 80 is in a fixed position relative to tube 64, e.g., by virtue of the return electrode being glued and/or otherwise mechanically coupled to the tube. In other embodiments, the return electrode is moveable within the tube. Treatment electrode 68 is configured to attract the thrombus upon the application of a treatment signal between the treatment electrode and the return electrode.

Device 62 further comprises a first wire 82, configured to connect treatment electrode 68 to power source 46. For example, end cap 72 may be coupled to the treatment electrode, and first wire 82 may pass through first lumen 74 and distally connect to the end cap such that, upon the proximal end of the first wire being connected to power source 46, the first wire connects the treatment electrode, via the end cap, to the power source. In some such embodiments, end cap 72 comprises a plug 88 that protrudes into, and plugs, first lumen 74, and the first wire is distally connected to plug 88.

In some embodiments, first wire 82 passes through return electrode 80; for example, the return electrode may comprise a metallic tube, and the first wire may pass through the lumen of the metallic tube. In other embodiments, the first wire passes through first lumen 74 alongside the return electrode.

The first wire is typically made of a biocompatible metal such as stainless steel, nitinol, tungsten and/or titanium. Typically, only the distal end of the first wire, which is connected to the treatment electrode (e.g., via end cap 72), is exposed, while the remainder of the first wire is insulated by an insulating layer 86 of a biocompatible material such as a polyimide or silicone. Typically, insulating layer 86 protrudes from return electrode 80 for at least 1 mm, such as at least 4 mm, at least while the treatment signal is applied. Thus, the flow of electric current between the more distal exposed portion of first wire 82 and the return electrode is reduced.

Device 62 further comprises a second wire 84, configured to connect return electrode 80 to power source 46. Typically, second wire 84 is entirely external to tube 64.

Alternatively to power source 46, the treatment electrode and return electrode may be connected to another power source via the first and second wires, as described above with reference to FIG. 6.

Typically, apertures 66 are aligned with first lumen 74. Thus, an electric current may flow between treatment electrode 68 and return electrode 80 via the apertures. In addition, a suctional force may be applied to the thrombus via the apertures.

In some embodiments, tube 64 is shaped to define one or more lateral windows 90 opening into second lumen 76. Typically, each window 90 is disposed near the distal end of the tube, e.g., between the proximal and distal ends of the treatment electrode, slightly proximal to (e.g., within 50 mm of) the proximal end of the treatment electrode, or slightly distal to (e.g., within 50 mm of) the distal end of the treatment electrode. In some embodiments, one or more windows 90 are located between successive windings or successive segments of the treatment electrode. Each window 90 may be slit-shaped, circular, or may have any other suitable shape.

Following the impedance measurements, thrombectomy device 62 is advanced over electrode assembly 23 while the electrode assembly is disposed within second lumen 76. Subsequently, during the thrombectomy procedure, one or more tools, and/or a contrast agent, may be passed through windows 90. Alternatively or additionally, as the thrombectomy device is withdrawn, break 48 may be aligned with a window located between the proximal and distal ends of the treatment electrode (and hence, near the thrombus), and the impedance-indicating signal may be monitored. If a significant change in impedance is observed, it is possible that part of the thrombus became detached from the treatment electrode; hence, the thrombectomy may be repeated.

Although FIGS. 6-7 show the embodiment of electrode assembly 23 shown in FIG. 4, it is noted that any suitable embodiment of the electrode assembly, such as that shown in FIG. 5, may guide the thrombectomy device.

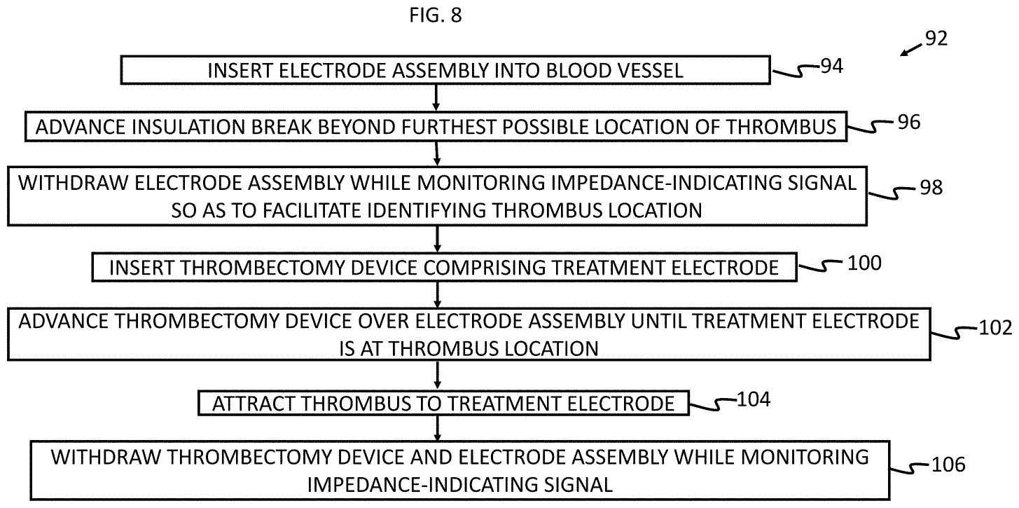

Reference is now made to FIG. 8, which is a flow diagram for a method 92 for treating a thrombus, in accordance with some embodiments of the present invention.

Method 92 begins with an inserting step 94, at which electrode assembly 23 (FIGS. 4-5) is inserted into a blood vessel of a subject.

Subsequently to the insertion of the electrode assembly, the electrode assembly is moved through the blood vessel while the impedance-indicating signal between the inner and outer electrodes of the electrode assembly, which results from ions (i.e., electrical current) flowing between the electrodes via at least one break in the insulating cover, is monitored.

For example, the break may be advanced beyond the furthest possible location of the thrombus at an advancing step 96. (In the case of multiple breaks, such as multiple perforations, the most proximal break may be advanced beyond the furthest possible location of the thrombus.) Subsequently, at a withdrawing step 98, the electrode assembly may be withdrawn while the impedance-indicating signal is monitored. Alternatively, the impedance-indicating signal may be monitored as the electrode assembly is advanced. In either case, monitoring the impedance-indicating signal facilitates identifying the location of the thrombus, given that the impedance-indicating signal typically indicates a change in impedance when the insulation break is at this location.

Subsequently, the thrombus is treated responsively to the impedance-indicating signal.

For example, a thrombectomy device comprising a treatment electrode, such as the thrombectomy device shown in FIG. 6 or FIG. 7, may be inserted into the blood vessel at another inserting step 100. Subsequently, at another advancing step 102, the thrombectomy device may be advanced over the electrode assembly until the treatment electrode is at the location of the thrombus. Subsequently, the thrombus is attracted to the treatment electrode at an attracting step 104. To attract the thrombus, a treatment signal is applied between the treatment electrode and an electrically-conductive element disposed within the thrombectomy device. For example, the treatment signal may be applied between the treatment electrode and one of the electrodes belonging to the electrode assembly, as described above with reference to FIG. 6, or between the treatment electrode and return electrode 80, as described above with reference to FIG. 7.

Finally, at a withdrawing step 106, the thrombectomy device (together with the thrombus) and the electrode assembly are withdrawn while the impedance-indicating signal is monitored. To facilitate this monitoring, the insulation break(s) may be aligned with apertures 66 (FIG. 6) or with a lateral window 90 disposed between the proximal and distal ends of the treatment electrode (FIG. 7) during the withdrawal.

It will be appreciated by persons skilled in the art that the present invention is not limited to what has been particularly shown and described hereinabove. Rather, the scope of embodiments of the present invention includes both combinations and subcombinations of the various features described hereinabove, as well as variations and modifications thereof that are not in the prior art, which would occur to persons skilled in the art upon reading the foregoing description. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. An electrode assembly, comprising:
an electrically-conductive wire, configured for insertion into a blood vessel of a subject;
an electrode surrounding the wire; and
a discontinuous electrically-insulating cover disposed between the wire and the electrode such that the wire lies radially opposite the electrode at a break in the discontinuous electrically-insulating cover.

2. The electrode assembly according to claim 1, wherein the electrode is shaped to define a helix.

3. The electrode assembly according to claim 1, wherein the electrode comprises a mesh.

4. The electrode assembly according to claim 1, wherein the electrode is coupled to the discontinuous electrically-insulating cover.

5. The electrode assembly according to claim 1, wherein a length of the break is less than 5 mm.

6. The electrode assembly according to claim 1, further comprising a reinforcing tube disposed around the discontinuous electrically-insulating cover.

7. The electrode assembly according to claim 6, wherein the reinforcing tube is proximal to the break.

8. The electrode assembly according to claim 6, wherein the reinforcing tube is distal to the break.

9. The electrode assembly according to claim 6, further comprising another electrode coupled to the reinforcing tube.

10. The electrode assembly according to claim 1, wherein the break includes a perforation in the discontinuous electrically-insulating cover.

11. The electrode assembly according to claim 10, wherein a surface area of the perforation is less than 0.8 mm².

12. Apparatus, comprising:
the electrode assembly according to claim 1; and
a treatment device, comprising:
a treatment element configured to treat an occlusion in the blood vessel; and
an electrically-insulating tube configured to facilitate delivery of the treatment element to the occlusion by advancing over the electrode assembly.

13. The apparatus according to claim 12,
wherein the occlusion includes a thrombus,
wherein a distal portion of the electrically-insulating tube is shaped to define one or more apertures, and
wherein the treatment element comprises a treatment electrode surrounding the distal portion of the electrically-insulating tube and configured to attract the thrombus when a signal is applied between the treatment electrode and any electrically-conductive element disposed within the electrically-insulating tube, by virtue of an electrical current flowing through the apertures.

14. The apparatus according to claim 13,
wherein the electrically-insulating tube is shaped to define a first lumen and a second lumen,
wherein the treatment element further comprises a return electrode,
wherein the signal is applied between the treatment electrode and the return electrode while the return electrode is disposed within the first lumen, and
wherein the electrically-insulating tube is configured to advance over the electrode assembly while the electrode assembly is disposed within the second lumen.

15. The apparatus according to claim 14, wherein the electrically-insulating tube is shaped to define a lateral window opening into the second lumen and disposed between a proximal end of the treatment electrode and a distal end of the treatment electrode.

16. A method, comprising:
inserting, into a blood vessel of a subject, an electrode assembly including:
a wire,
an electrode surrounding around the wire, and
a discontinuous electrically-insulating cover disposed between the wire and the electrode such that the wire lies radially opposite the electrode at a break in the discontinuous electrically-insulating cover;
subsequently to inserting the electrode assembly, moving the electrode assembly through the blood vessel while monitoring a signal between the electrode and the wire, which results from ions flowing between the wire and the electrode via the break; and
treating an occlusion in the blood vessel responsively to the signal.

17. The method according to claim 16, wherein treating the occlusion comprises:
responsively to the signal, positioning a treatment device, which includes an electrically-insulating tube, by advancing the electrically-insulating tube over the electrode assembly; and
using the treatment device, treating the occlusion.

18. The method according to claim 17,
wherein the occlusion includes a thrombus,
wherein the signal is a first signal,
wherein a distal portion of the electrically-insulating tube is shaped to define one or more apertures,
wherein the treatment device further includes a treatment electrode surrounding the distal portion of the electrically-insulating tube such that the apertures are disposed between a proximal end of the treatment electrode and a distal end of the treatment electrode, and
wherein treating the thrombus comprises attracting the thrombus to the treatment electrode by applying a second signal between the treatment electrode and any electrically-conductive element disposed within the electrically-insulating tube, by virtue of an electric current flowing through the apertures.

19. The method according to claim 18, wherein treating the thrombus further comprises:
subsequently to a start of the second signal, withdrawing the electrically-insulating tube and the electrode assembly from the blood vessel while the break in the discontinuous electrically-insulating cover is aligned with at least one of the apertures; and
while withdrawing the electrically-insulating tube and the electrode assembly, monitoring the first signal.

20. The method according to claim 18, wherein the electrically-conductive element is selected from the group of elements consisting of: the wire, the electrode, and another electrically-conductive element disposed over the wire.

21. The method according to claim 18,
wherein the electrically-insulating tube is shaped to define a first lumen and a second lumen,
wherein the apertures open into the first lumen,
wherein the electrically-conductive element includes a return electrode disposed within the first lumen, and
wherein advancing the electrically-insulating tube over the electrode assembly comprises advancing the electrically-insulating tube over the electrode assembly while the electrode assembly is disposed within the second lumen.

22. The method according to claim 21, wherein the electrically-insulating tube is shaped to define a lateral window opening into the second lumen and disposed between a proximal end of the treatment electrode and a distal end of the treatment electrode, and wherein treating the thrombus further comprises:
subsequently to a start of the second signal, withdrawing the electrically-insulating tube and the electrode assembly from the blood vessel while the break in the discontinuous electrically-insulating cover is aligned with the lateral window; and
while withdrawing the electrically-insulating tube and the electrode assembly, monitoring the first signal.

23. The method according to claim 16,
wherein the occlusion includes a thrombus,
wherein the signal is a first signal, and
wherein treating the thrombus comprises:
positioning the electrode assembly responsively to the first signal; and
attracting the thrombus to the electrode by applying a second signal between the electrode and the wire.

24. The method according to claim 16, further comprising generating the signal by applying a voltage or current between the electrode and the wire.

25. The method according to claim 16,
wherein the electrode assembly further includes a pair of other electrodes, and
wherein the method further comprises generating the signal by applying a voltage or current between the pair of other electrodes.
